(19) United States
(12) Patent Application Publication
(10) Pub. No.: US 2007/0235665 A1
(43) Pub. Date: Oct. 11, 2007

(54) Charged particle beam system and method for manufacturing and inspecting LCD devices

(75) Inventors: Bassaam Shamoun, Fremont, CA (US); Fayez E. Abboud, Pleasanton, CA (US)

Correspondence Address: PATTERSON & SHERIDAN, LLP, 3040 POST OAK BOULEVARD, SUITE 1500, HOUSTON, TX, 77056, US

(73) Assignee: Applied Materials, Inc.

(21) Appl. No.: 11/393,290

(22) Filed: Mar. 30, 2006

(51) Int. Cl. A61N 5/00; G21K 5/10

(52) U.S. Cl. 250/492.23; 250/492.2

(57) ABSTRACT

A layer on a large area substrate is patterned by providing a large area substrate in the optical path of a plurality of charged particle beams that are emitted from charged particle emitters. Each charged particle beam has an emitted beam current of at least I₀, and the beam current on the substrate is at least 0.4 I₀. Each charged particle beam is deflected in at least one dimension and is switched on and off to generate an exposed pattern on the photoresist film.

### BACKGROUND OF THE INVENTION

1. Field of the Invention

Embodiments of the invention generally relate to a charged particle beam system for large area substrates and a method of operating the system, and more specifically, embodiments of the invention relate to a charged particle beam system for manufacturing an LCD color filter substrate and other large area substrate devices and method of operating the system.

2. Description of the Related Art

Flat panel displays, such as active matrix liquid crystal displays, have recently become commonplace in the world as a replacement for the cathode ray tubes of the past. A liquid crystal display (LCD) has several advantages over the CRT, including higher picture quality, lighter weight, lower voltage requirements, and low power consumption. The displays have many applications in computer monitors, cell phones and televisions to name a few.

One type of active matrix LCD includes a liquid crystal material sandwiched between a thin film transistor array (TFT) substrate and a color filter substrate to form a flat panel substrate. The TFT substrate includes an array of thin film transistors, each connected to a pixel electrode and the color filter substrate includes different color filter portions. When a certain voltage is applied to a pixel electrode, an electric field is created that orients the liquid crystal material to allow light to pass therethrough for that particular pixel.

The demand for larger displays, increased production and lower manufacturing costs has created a need for new manufacturing systems that can accommodate larger substrate sizes. Current TFT LCD processing equipment is generally configured to accommodate substrates up to about 1500 mm×1800 mm (i.e., a surface area of about 25,000 cm²) or sizes up to and exceeding 1900 mm×2200 mm (i.e., greater than 40,000 cm²). Current equipment may even accommodate substrates up to about 2200 mm×2400 mm (i.e., greater than 50,000 cm²) and larger. Generally, substrates from about 1100 mm×1250 mm (i.e., greater than 15,000 cm²) to about 2200 mm×2400 mm (i.e., greater than 50,000 cm²) and larger may be considered large area substrates. The size of the processing equipment as well as the process throughput time is a great concern to flat panel display manufacturers, both from a financial standpoint and a design standpoint.

As a part of the manufacturing process, photoresist patterning has to be conducted for processing desired structures on the substrate, e.g., to selectively etch the desired structures. Presently, a costly mask is employed in this process. The mask is exposed with a laser tool to pattern a photoresist on the substrate so as to allow selective etching of the substrate using the patterned photoresist. Costs of the equipment in form of an electron beam writing system, for producing the mask, and the mask itself are high.

Mask-less photoresist patterning using electron beam direct write systems with sufficient throughput have been proposed for small area substrates. The attempt to meet throughput requirements was based on a high number of electron beams, e.g., in the order of 100 electron beams and above. As one example, axis-free systems have been proposed in order to integrate more charged particle beams. In such a system, several charged particle beams are imaged within one charged particle optic. As another example, electron beam direct write systems with closely spaced mini-columns, which are defined as having column housings allowing spacing between optical axes of neighboring columns below 100 mm, have been proposed. A further increase of the number of electron beams to meet the throughput requirements of large area substrates is costly and may have structural constraints.

### SUMMARY OF THE INVENTION

The invention generally provides a method manufacturing large area substrate devices that employs a novel technique for of patterning a layer on large area substrates. The novel technique, according to one embodiment, includes providing a large area substrate in the optical path of charged particle beams from a plurality of charged particle emitters, wherein each charged particle beam has an emitted beam current of at least I₀, and the beam current on the substrate is at least 0.5 I₀. Each charged particle beam is deflected in at least one dimension and is switched on and off to generate an exposed pattern on the photoresist film.

The invention also provides a method of manufacturing a color filter. The method according to an embodiment includes providing a large area substrate with a black matrix layer for a color filter in a chamber of a charged particle beam system, exposing a photoresist on the black matrix layer with the charged particle beam system, wherein less than 14 charged particle beams are scanned over the photoresist, and etching recesses in the black matrix layer, and filling color filter materials corresponding to colors of the color filter in the recesses etched in the black matrix layer.

The invention also provides a charged particle beam system for manufacturing devices on large area substrates. The system includes a chamber for receiving the large area substrate, a plurality of charged particle beam generators arranged in an array and fixed to the chamber, and a plurality of charged particle beam guiding optics, each corresponding to one of the charged particle beam generators and each having an emitter, a grid, an anode with an aperture and at least two lenses, wherein each of the charged particle beam optics is configured to deliver at least 50% of the charged particles emitted from the emitter to the large area substrate.

The invention is also directed to apparatuses for carrying out the disclosed methods, including apparatus parts for performing each of the described method steps. These method steps may be performed by way of hardware components, a computer programmed by appropriate software, by any combination of the two, or in any other manner. Furthermore, the invention is also directed to methods by which the described apparatus operates or is manufactured. It includes method steps for carrying out every function of the apparatus.

### BRIEF DESCRIPTION OF THE DRAWINGS

So that the manner in which the above recited features of the invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustrates a schematic view of a section of an LCD flat panel display.

FIG. 2 illustrates a schematic top view of a section of a color filter that may be used for a LCD flat panel display.

FIG. 3A is a flow chart illustrating a method of an embodiment according to the invention.

FIG. 3B is a flow chart illustrating a method of a further embodiment of the invention.

FIG. 4 illustrates a schematic side view of an embodiment of a charged particle beam device adapted for direct electron beam writing on large substrates.

FIG. 5 illustrates a schematic view of an embodiment of a beam path in a charged particle beam for explaining the beam characteristics used in embodiments of the invention.

FIG. 6 illustrates a schematic top view of an embodiment having eight columns for a large substrate system.

FIG. 7 shows a graph illustrating the dependency of beam current as a function of substrate throughput.

FIG. 8 is a picture of a patterned photoresist.

FIG. 9 illustrates a schematic view of one embodiment of an in-line system having two load lock chambers.

### DETAILED DESCRIPTION

The invention relates to charged particle beam writing on large area substrates, especially for patterning a black matrix of color filters applied to LCD flat panel displays. The invention further relates to systems for charged particle beam writing, with charged particle beam characteristics adapted for writing on large area substrates, especially on a black matrix of color filters applied to LCD flat panel displays. Expensive masks and expensive processes including exposing the mask may be eliminated.

Without limiting the scope of protection of the present application, the charged particle beam device will be referred to herein as an electron beam device. The electron beam device might be used in an electron beam inspection or lithography system. The invention may be used with other sources of charged particles and/or other secondary and/or backscattered charged particles.

Those skilled in the art will also appreciate that all discussions herein related to voltages and potentials refer to relative and not absolute terms. For example, accelerating the beam by connecting an emitter to “ground” and applying 3 kV to the sample is equivalent to applying negative 3 kV to the emitter and placing the specimen on ground. Therefore, while some discussion is provided in terms of specific voltages, it should be understood that the reference is to relative potential.

LCD displays may generally be described with regard to three parts. First, there is an illumination section for providing a back-light or the like. Second, there is an LCD section providing controllable electrodes to provide a potential to the LCD material at individual sub-pixels. Third, in the case of a color display, there may be a color filter, to provide 3 or 4 different colors for adjacent sub-pixels. The differently colored sub-pixels form a “white” pixel, the color of which may be controlled by controlling the relative potentials of the sub-pixels.

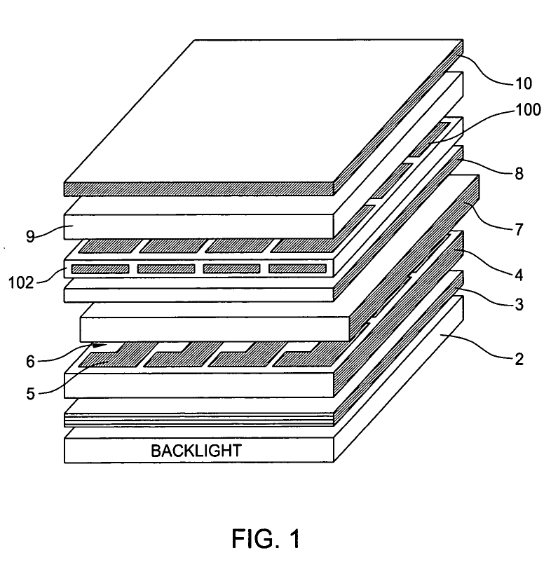

FIG. 1 illustrates an embodiment of an LCD display. A backlight 2 is provided below a polarizer 3. Electrodes 5 are provided on a glass substrate 4. The optical properties of the areas in the liquid crystal layer 7, which correspond to the electrodes 5, are varied by applying a potential between individual of the electrodes 5 and the common top electrode 8. In one embodiment, individual electrodes 5 are addressed e.g. by thin film transistors (TFT) 6, as shown in the active matrix LCD of FIG. 1. In another embodiment, the electrodes may be formed by overlapping regions of bottom electrode lines and essentially perpendicular top electrode lines. Thereby, a passive matrix LCD may be provided.

The transmissibility of individual picture elements for the backlight is changed depending on the potential difference between individual electrodes 5 and the top electrode 8. The potential difference induces a change of the polarization in the LCD material of the liquid crystal layer 7. The polarization changes result in a changing light intensity from the backlight 2, through the polarizer 3, the glass plate 9 and the polarizer 10. As a result, an image can be generated by applying charge to the individual pixel electrodes, accordingly.

A color LCD display typically includes a color filter array 100. Therein, for example, three different color filter materials are provided in a black matrix 102. The color filter materials may be dyes for red, green, and blue (RGB). Within FIG. 1, the colors are arranged to have red dyed areas, green dyed, and blue dyed areas in a diagonal pattern.

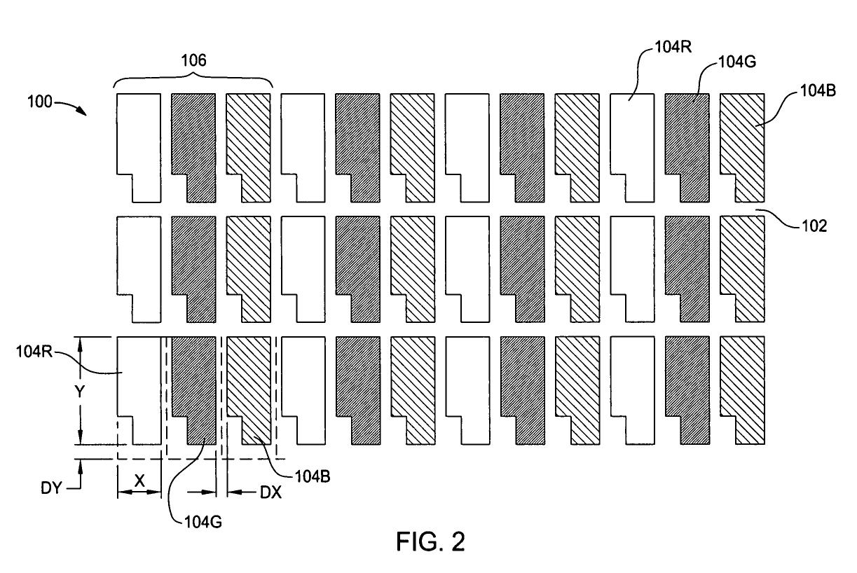

FIG. 2 is a more detailed view of an embodiment of a color filter. The filter areas 104R, 104G, and 104B correspond to electrodes capable of controlling the intensity of transmitted or reflected light. The filter areas form sub-pixels, which are the smallest individual controllable area and which are given by an electrode area. A red dyed area 104R, a green dyed area 104G, and a blue dyed area 104B form one pixel 106 as visualized by an observer of a display. In light thereof, even though the electrodes 5, as illustrated in FIG. 1, are often referred to as pixel electrodes, they should, for a color display, better be denoted as sub-pixel electrodes.

The dyed filter areas 104R, 104G, and 104B are formed in a black matrix 102. The black matrix may contain polyimide or other suitable materials, such as metal or resin, which are capable of shielding scattered light. Within the embodiment of FIG. 2, the length, i.e., the vertical dimension of a sub-pixel, is denoted as Y and the width, i.e., the horizontal dimension of a sub-pixel, is denoted as X. The vertical distance between adjacent sub-pixels is denoted as dY and the horizontal distance is denoted as dX. In one embodiment, the pitch of the pixels 106 may be (X+dX) 295 μm and (Y+dY) 295 μm. Corresponding dimensions may be X=90 μm, dX=10 μm, Y=285 μm and dY=10 μm. This results in a coverage of the black matrix 102 of about 10%. A coverage of 10% may convert for a glass substrate size of 1850 mm to 2200 mm to an area of the black matrix of 407000 mm². In another embodiment with a coverage of about 20%, the corresponding black matrix area is about 814000 mm². According to one embodiment, a color filter may be characterized as having a black matrix coverage between 5% and 15%. Generally, the substrates may, according to one embodiment, be characterized as being rectangular.

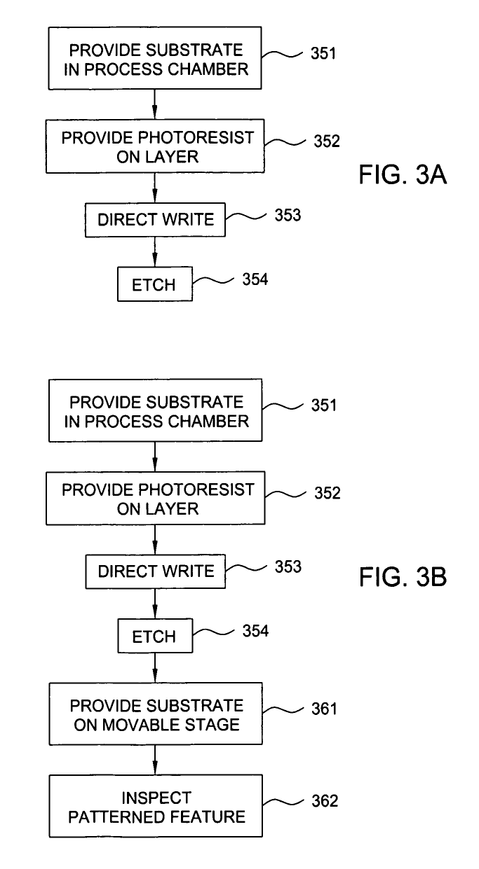

FIG. 3A sets forth the steps in processing a large area substrate according to one embodiment of the invention. In step 351, the substrate having the layer to be patterned is provided in a processing chamber. According to one embodiment, the layer is a black matrix layer made of polyimide, for example. Within the processing chamber, a photoresist layer is provided on the layer in step 352. In step 353, the photoresist layer is exposed by writing directly on the photoresist layer with an electron beam. In this step, the electron beams are scanned over a plurality of direct write areas on the photoresist layer. These deflection areas have, according to one embodiment, a size of more than 280 mm×280 mm. Typically, the size may be 320 mm×320 mm. A plurality of electron beams is provided, each for a direct write area. Each electron beam may be scanned over its respective area by being deflected in two directions that are mutually orthogonal and substantially perpendicular to its optical axis For example if the optical axis of the electron beam column is the z-axis, the electron beam is deflected in the x-direction and the y-direction. According to an alternative embodiment, each electron beam is deflected in the y-direction, and the relative movement of the electron beam and the substrate in the x-direction is caused by an x-movement of the substrate.

According to one embodiment, the deflection of the electron beams of the multiple electron beam columns may be synchronized. However, it is understood, that the electron beams of the multiple electron beam columns may also be deflected independently of each other.

During deflection of an electron beam over a direct write area, the electron beam is switched on and off with a blanking unit. The electron beam is switched on over portions of the direct write area that are supposed to be exposed by the electron beam. The electron beam is switched off over the other portions. Thereby, the desired exposure pattern is generated.

Step 353 may be conducted while the substrate is maintained in the processing chamber, or the substrate may be moved to a different chamber having an exposure tool before step 353 so that the direct writing in accordance with step 353 may be carried out in the different chamber. After step 353, the substrate is moved to an etching chamber after step 353. In step 354, the substrate is etched to generate the desired pattern in the layer, e.g., the polyimide layer. After the etching, the photoresist layer is removed before further manufacturing steps.

A further advantage of the invention is illustrated by the method shown in FIG. 3B. Steps 351 to 354 are similar to the embodiments described with respect to FIG. 3A and a repetition thereof is omitted for simplicity. In step 361, the patterned black matrix of the color filter is provided on a movable stage below the above-described electron beam columns. The stage moves the substrate such that the electron beams can be scanned over the deflection areas for purposes of inspecting the etched features. The inspection (step 362) is conducted by detecting the secondary or backscattered electrons released from the substrate upon impingement of the electron beams. The intensities detected by, e.g., a scintillation detector or the like, can be evaluated to determine the composition, the topography, or other characteristics known in the art. The embodiment illustrated in FIG. 3B allows the electron beam direct write tool to be applied for inspection. As a result, prior art light optical measurement methods to inspect the patterned black matrix can be avoided. An inspection of the black matrix may be important to the process of manufacturing the color filter, since any defects in the patterned black matrix may deteriorate the filling of the color filter material in the etched recesses. This may be especially relevant if the color filter materials are injected in the recesses with an ink-jet method.

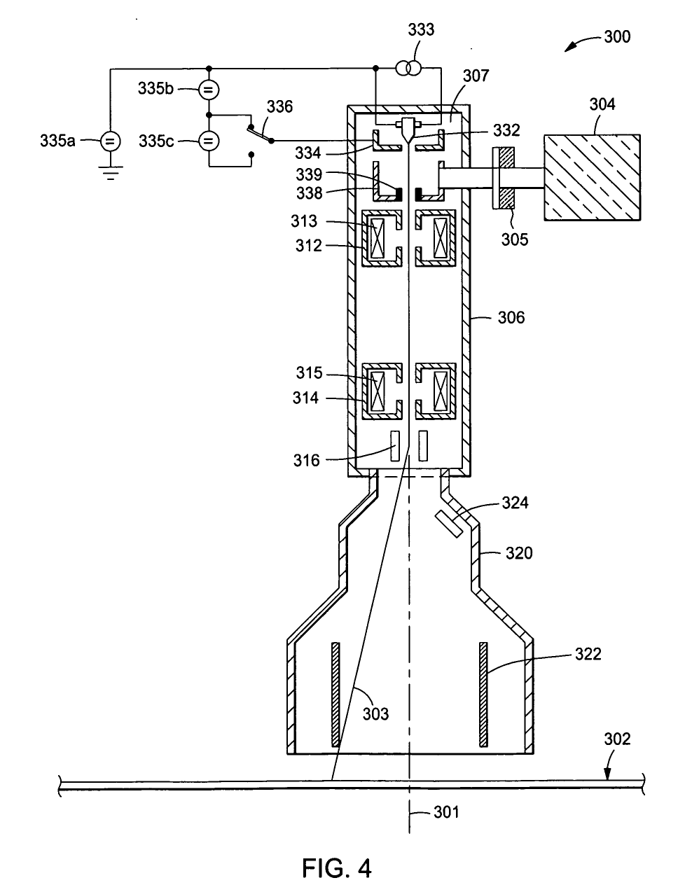

An embodiment of a charged particle beam column, which may be used for any of the processes shown in FIG. 3A or FIG. 3B, i.e. for testing or writing, is shown in FIG. 4. The optics in electron beam column 300 guides electron beam 303 on the target 302. The electron beam is emitted by the electron emitter 332, such as a LaB₆ emitter. The beam current and the shape of a first crossover can be controlled by the Wehnelt grid 334. The beam energy and the beam shape may be controlled by the beam shaping aperture 339 in an anode 338. A condenser lens 312 including coil 313 and an objective lens 314 including coil 315, which are included in the housing 306, image the electron beam. The lens may be magnetic, electrostatic, or combined electrostatic-magnetic. In one embodiment, the objective lens 314 includes a main focusing unit and a sub-focusing unit. The sub-focusing unit, which may for example either be magnetic or electrostatic, is used for small adjustments, since the main focusing unit may not be able to provide fast corrections in light of the electromagnetic induction in the coil.

A deflection system 316, such as an electrostatic deflection system, a magnetic deflection system, or a combination thereof, deflects the electron beam of the optical axis 301 and guides the electron beam onto a location on target 302. On impingement of the primary electron beam 303 onto the target 302, secondary or backscattered electrons, photons or X-rays are released. These particles, which are herein generally referred to as secondary particles, are guided to detector 324 by guiding electrodes 322, both included of a lower part 320 of the column. Detector 324 may detect the secondary particles released from the location of impingement of the primary electron beam 303.

The gun area 307 may typically be a separate vacuum chamber, which can be evacuated by vacuum pump 304 via valve 305. The pump may, e.g., be an ion getter pump. According to one embodiment, the beam emission is controlled as follows. Current source 333 heats emitter 332 by providing a current. The emitted beam current can be controlled, amongst other things, by the temperature which may be between about 1100 K and 1400 K. Typically the temperature of the emitter 332 acting as the cathode may be about 1250K. Grid voltage source 335b applies an extracting voltage of 500 V to 900 V, typically 700 V, which extracts electrons from the cathode. Grid 334, also named Wehnelt grid, further focuses the electron beam to form a first crossover.

Blanker voltage source 335c can be connected to grid 334 with switch 336. Thereby, the grid voltage may be changed by about 200 V, or the like. Decreasing the grid voltage by 200 V reduces the extraction voltage to an amount such that no electrons are extracted from the cathode, i.e., emitter 332. Thus, switch 336 may be used to switch between electron emission and no electron emission. Switch 336 and blanker voltage source 335c form a blanker unit for blanking the emission of electrons from the emitter 332.

The primary energy, of the electron beam with respect to the target is controlled by primary energy voltage source 335a. In the embodiment shown in FIG. 4, the target 302 and the anode 338 may be at ground potential. The electrons are emitted and accelerated towards the anode to an energy corresponding to the voltage of primary energy voltage source 335a. After passing through the aperture 339 and anode 338, the electrons impinge on target 302 with the energy corresponding to the voltage of the primary energy voltage source. The primary energy may be in the range of 1 keV to 30 keV, typically 10 keV.

Typical substrate size dimensions are the 15 k, 25 k, 40 k, and 50 k generations relating to dimensions as follows: substrate dimensions of 1.1 meters×1.25 meters (i.e., greater than 15,000 cm²), substrate dimensions of 1.5×1.8 meters (i.e., a surface area of about 25,000 cm²), substrates dimensions of 1.9×2.2 meters (i.e., greater than 40,000 cm²), or substrate sizes of 2.2 meters×2.4 meters (i.e., greater than 50,000 cm²). Direct write systems capable of providing a throughput adequate for industrial applicability, which may be in range from 1 minute to 8 minutes, have not been considered to be realized with charged particle beam devices as, e.g., shown in FIG. 4.

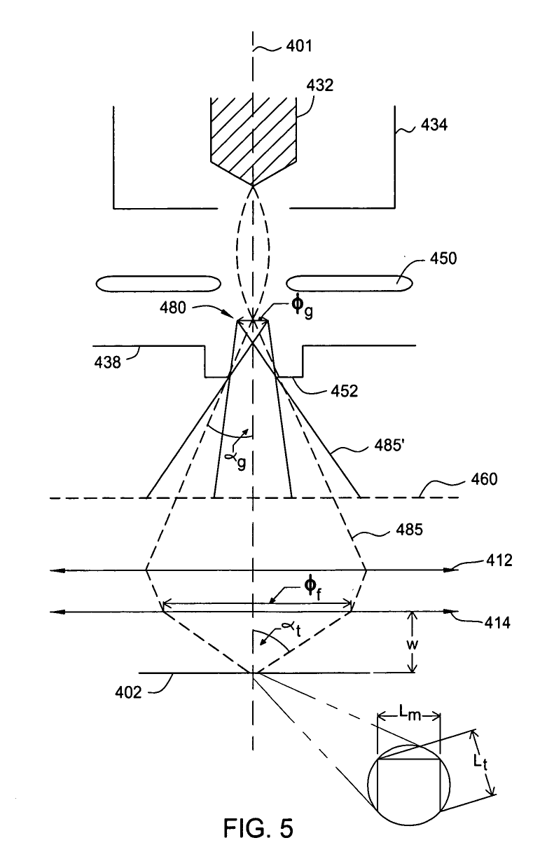

According to an embodiment of the invention, a direct write charged particle beam system can be realized for LCD substrates of the 15 k, 25 k, 40 k, and even 50 k generation with eight to ten charged particle beam columns. Embodiments of beam parameters of an electron beam system are described with respect FIG. 5. FIG. 5 shows an embodiment with a LaB₆ electron emitter 432. Grid 434 (Wehnelt) extracts electrons and forms a first crossover 480, as indicated by the dashed lines 485. Generally, dashed lines 485 illustrate the beam path of the electron beam along optical axis 401 through the system including a shield 450, an anode 438 with aperture 452, first lens 412 (condenser), second lens 414 (objective), and target 402. The size Φ_{g} of the crossover an the resulting beam path 485′ are also indicated in FIG. 5. Beam path 485′ shows an image on a screen 460, which may be positioned to evaluate the beam parameters, particularly in the far field.

Within FIG. 5, D denotes the distance between the crossover 480 and the condenser lens 412, α_{g} denotes the beam divergence semiangle, αₜ denotes the beam semiangle from objective lens 414 to target 402, Φ_{g} denotes the beam diameter in the objective lens, Lₜ denotes the illumination length and Lₘ denotes the flash size.

The individual beam parameters are related to each other by Helmholtz-Lagrange theory, appreciated by a person of skill in the art. Herein, details regarding established theories are omitted for simplicity. Generally, a direct write electron beam system as exemplarily shown in FIG. 5 can be evaluated by the current density at the target to predict the throughput, because the photoresist to be exposed has a given dose requirement, i.e., a given amount of charged per area that is required to develop the photoresist. The electron beam current density at the target is, according to Helmholtz-Lagrange, a function of the brightness of the emitter (units: A/cm² sr), the emittance (units: μm mrad), and the illumination area on the target.

Previously, the proposed maskless charged particle beam systems used beam parameters to allow for good imaging quality, which results in a good resolution, i.e. a small pot size on the target. Dose requirements have been proposed to be met by increasing the number of electron beams in the order of hundreds and above. This may be better understood considering the following influences. An electron beam with a large brightness of the emitter and a small emittance Φ_{g}α_{g} has a high electron density within the beam traveling through the column. The charged electrons are subject to repelling forces with respect to each other. This effect is referred to as Boersch effect or space charge error in the background art literature. The Boersch effect results in a broadening of the diameter of the beam in the column and results in an increase of the energy width of the electron beam, because some of the electrons may be accelerated by adjacent electrons whereas some of the electrons may be decelerated by adjacent electrons. The increasing energy width of the beam then results in increased chromatic aberrations.

To decrease the electron density in the beam, the emittance Φ_{g}α_{g} may be increased. This, however, results in an increase of spherical and chromatic aberrations. As a result, in the past, it has generally be propose to increase the number of beams. Increasing the number of beams allows the electron density within the charged particle beam to be reduced for each beam, which also reduces the space charge error. Parts of the electron beam are for example blocked by the aperture in the anode. Previously, the brightness has been reduced by blocking about 70% to 90% of the emitted electrons. About 10% to 30% of the emitted brightness has been used for irradiation of the target

Contrary thereto, embodiments of the invention realize a trade-off between beam quality and current density on the target. It has been found that this approach allows for realizing systems with appropriate throughput and writing quality.

According to one embodiment, a flash size between 2 μm and 8 μm is used. Applying this flash size to a desired throughput of about 3 minutes to about 9 minutes, a 1850 mm to 2200 mm substrate size for a color filter with 10% black matrix coverage, as described with respect to FIG. 2, a system with 8 columns and a dose requirement for the photoresist of 10 μC/cm², a beam current density of about 14 A/cm² to about 706 A/cm² may be required, which results in a beam current per column of about 9 μA to about 28 μA. Higher current densities or currents, of course, also result in the above-mentioned maximal process times per substrate. According to one embodiment, applying flash size of 4 μm to a desired throughput of typically 5 minutes, a 1850 mm to 2200 mm substrate size for a color filter with 10% black matrix coverage, as described with respect to FIG. 2, a system with 8 columns and a dose requirement for the photoresist of 10 μC/cm², a beam current density of about typically 105 A/cm² or higher, may be required, which results in a beam current per column of about 17 μA or higher.

According to another embodiment, a flash size between 2 μm and 8 μm is used. Applying this flash size to a desired throughput of about 3 minutes to about 9 minutes, a 1850 mm to 2200 mm substrate size for a color filter with 10% black matrix coverage, as described with respect to FIG. 2, a system with 10 columns and a dose requirement for the photoresist of 10 μC/cm², a beam current density of about 11 A/cm² to about 565 A/cm² may be required, which results in a beam current per column of about 8 μA to about 23 μA. Higher current densities or currents, of course, also result in the above-mentioned maximal process times per substrate. According to one embodiment, applying flash size of 4 μm to a desired throughput of typically 5 minutes, a 1850 mm to 2200 mm substrate size for a color filter with 10% black matrix coverage, as described with respect to FIG. 2, a system with 10 columns and a dose requirement for the photoresist of 10 μC/cm², a beam current density of about typically 84 A/cm² or higher, may be required, which results in a beam current per column of about 14 μA or higher

According to a further embodiment, a flash size between 2 μm and 8 μm is used. Applying this flash size to a desired throughput of about 3 minutes to about 9 minutes, a 1850 mm to 2200 mm substrate size for a color filter with 15% black matrix coverage, as described with respect to FIG. 2, a system with 10 columns and a dose requirement for the photoresist of 5 μC/cm², a beam current density of about 8 A/cm² to about 424 A/cm² may be required, which results in a beam current per column of about 6 μA to about 17 μA. Higher current densities or currents, of course, also result in the above-mentioned maximal process times per substrate. According to one embodiment, applying flash size of 4 μm to a desired throughput of typically 6 minutes, a 1850 mm to 2200 mm substrate size for a color filter with 15% black matrix coverage, as described with respect to FIG. 2, a system with 10 columns and a dose requirement for the photoresist of 5 μC/cm², a beam current density of about typically 63 A/cm² or higher, may be required, which results in a beam current per column of about 10 μA or higher.

Given the source maximum brightness, the design of the electron gun may be conducted starting with minimizing the square root of the squares of the different kind of aberrations as a function of the beam semiangle from the objective lens αₜ. Thereby, other parameters such as beam voltage, column length, Wehnelt bias, anode-condenser distance D, aperture diameter, and the like may also be varied for optimization of the system. Generally, the beam characteristic requirements are designed to be a trade-off between electron density on the target and the influence of aberrations. Thus, at least 40%, typically 50%, 60% or even 90% of the emitted electrons may be used for the electron beam on the target. However, care has to be taken that the beam requirements in light of introduced aberrations are met.

Typical emittance Φ_{g}α_{g} values may for example be in the range of 500 to 900 μm mrad, e.g., 700 μm mrad. Currents of above 10 μA per electron beam column may further be increased by increasing the temperature of the emitter.

The embodiments described above, realize a set of beam parameters that allow for a electron beam current density on the substrate surface sufficiently high to meet the throughput requirements while, at the same time, the beam quality is sufficiently good. The beam quality is, however, decreased with respect to prior art electron beam systems. The above described embodiments trade off a justifiable amount of beam quality to be able to meet the throughput requirements. The above described embodiments enable a throughput increase that is not merely based on increase of the number of beams provided. Therefore, lower cost methods and systems may be realized.

A second important parameter to realize systems for direct write of LCD related substrates is the area which can be covered by one electron beam. The field of view of the electron beam, as denoted in testing systems, or the deflection area or field of writing, how it may be denoted for direct write systems, is according to one embodiment in the range of 280 mm×280 mm to 350 mm×350 mm. To enable a direct write systems for a 40k-substrate with for examples 8 or 10 columns, the field of writing may be above 300 mm×300 mm. Typically, it may be 320 mm×320 mm.

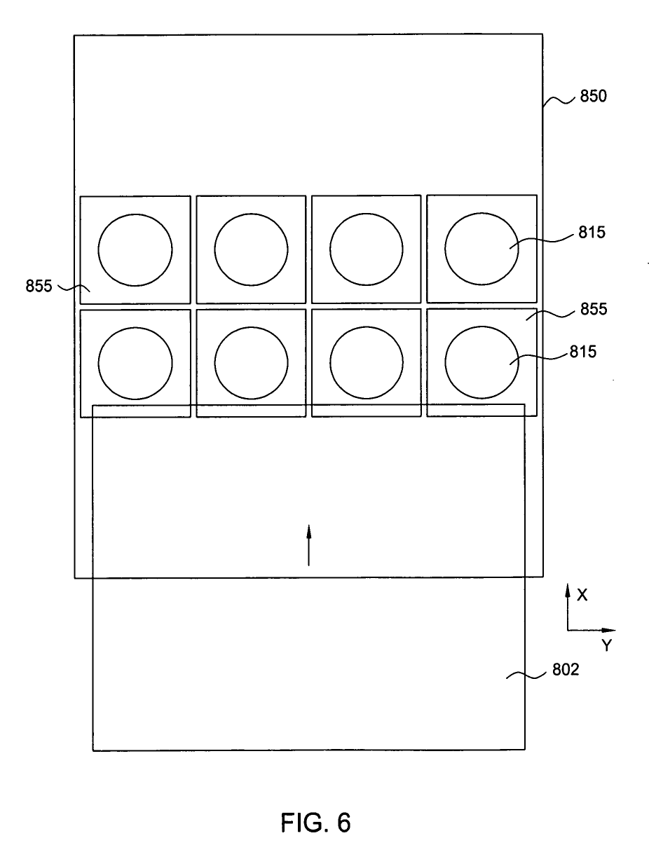

An embodiment of the arrangement of electron beam columns and the fields of writing is shown in FIG. 6. The embodiment of FIG. 6 has a chamber 850. On top of the chamber 850 there are eight electron beam columns 815. Each electron beam column 815 has deflection area 855, used as a field of view or a field of writing. These areas to which the electron beam can be deflected by a scanning deflector overlap. A substrate 802 which is inserted in chamber 850 can be measured by the electron beams.

According to one embodiment the electron beams of the eight columns are deflected in x-direction and in y-direction, respectively. After the area below the beams has been scanned, the substrate 802 is displaced and the next area including the eight fields of writing or view is patterned. According to another embodiment, the electron beams of the eight columns are deflected in y-direction, while the substrate 802 is moved in x-direction.

The deflection of the electron beam over the areas 855 on the target 802 may be conducted in two different modes. According to one embodiment, a raster writing mode is applied. Thereby, the electron beam is moved in x- and y-direction relative to the areas 855. The relative movement can be realized according to any of the above described methods. Within raster writing mode, the number of columns and spacing is determined based on the maximum deflection area 855 and available chamber space. The raster mode may be conducted with either a Gaussian beam shape or with a variable beam shape. Examples of variable beam shape methods and apparatuses that may be used are described in U.S. patent application Ser. No. 10/996,020, filed Nov. 22, 2004 entitled “METHOD FOR ELIMINATING LOW FREQUENCY ERROR SOURCES TO CRITICAL DIMENSION UNIFORMITY IN SHAPED BEAM WRITING SYSTEMS,” which is incorporated herein by reference to the extent it is not inconsistent with this disclosure.

According to another embodiment, which typically uses a variable beam shape, the beam may be directed on the target in a vector beam mode. The vector beam mode does not necessarily raster the entire substrate. This mode directs the beam directly to the vector of the next writing position. This may be advantageous if the pattern density is not uniform.

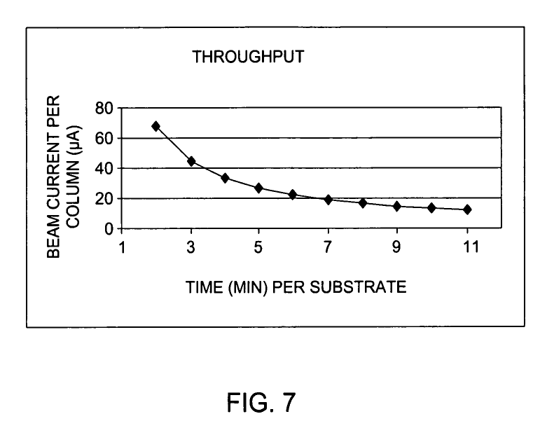

Calculated throughput data for the parameter ranges given above is shown in FIG. 7. An increase from 9 min/substrate to 4 min/substrate requires a beam current on the target per column from 15 μA to 34 μA, provided that a ten column system with a coverage of 20% of the substrate is given.

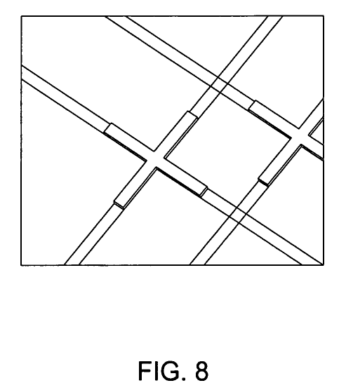

Preliminary test results on a system in which the beam parameter has been simulated by overwriting the same pattern several times are shown in FIG. 8. FIG. 8 shows that the electron beams may be used for direct writing on photoresist to provide for example structures in a color filter substrate for manufacturing of an LCD. Since throughput requirements can be met, the usage of two or more tools, like an electron beam writer for a mask and a laser for exposing the substrate with the mask can be avoided. Thereby, the cost of production as well as the overall throughput can be reduced by reducing the overhead time required by two stand-alone tools.

In addition, the direct write system provides an improved flexibility for the patterns to be written. As the pattern can be varied from one substrate to the next substrate without providing a new mask.

Particularly in the case where the direct write is applied for patterning the black matrix of a color filter, a system is provided including a composition of deflection fields covering the entire substrate. This system can then be used for testing and inspection of the color filer, which can currently not be conducted by charged particle beam inspection methods covering a sufficient substrate size.

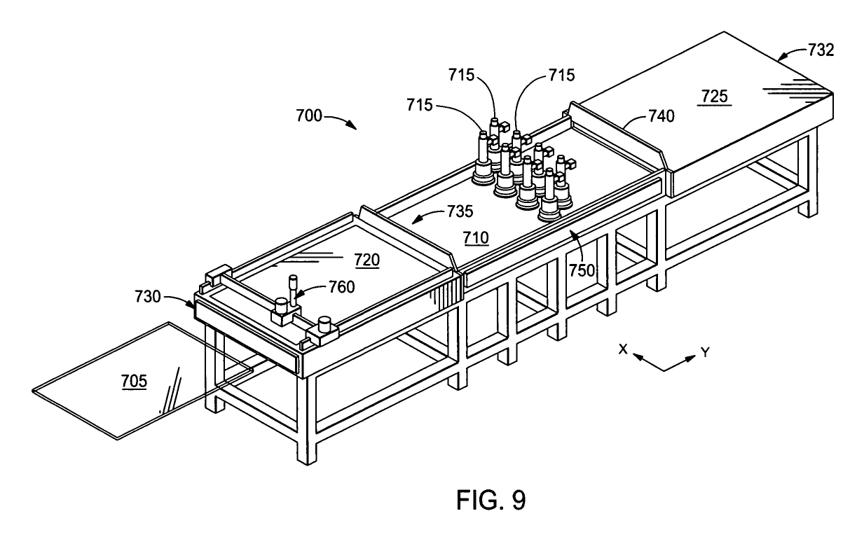

An embodiment of a write system, which may also be applied to color filter inspection, is shown in FIG. 9. FIG. 9 is an isometric view of one embodiment of an in-line system 700 adapted to write or test on large area flat panel substrates up to and exceeding about 2200 mm by 2600 mm. The system 700 includes a chamber 710, load lock chambers 720, 725, and electron beam columns 715 (eight are shown in FIG. 9). The system 700 is typically located in a clean room environment and may be part of a manufacturing system that includes substrate handling equipment such as robotic equipment or a conveyor system that transports flat panel substrates to and from the system 700. The load lock chambers 720, 725 are disposed adjacent and connected to the chamber 710 by slit valves 735, 740. The load lock chambers 720, 725 facilitate transfer of flat panel substrates to and from the chamber 710 and ambient environment. The load lock chamber 720 is adapted to receive a substrate from a clean room environment through an entry port 730, while the load lock chamber 725 has an exit port 732 that selectively opens to return the flat panel substrate to the clean room environment. The load lock chambers 720, 725 are sealable from ambient environment and are typically coupled to one or more vacuum pumps. The chamber 710 may be coupled to one or more vacuum pumps that are separate from the vacuum pumps of the load lock chambers 720, 725. The system 700 may also include a microscope 760 coupled to the system for review of any pixel defects encountered on the substrate. An example of various components of a large area system can be gathered from electron beam system for testing large area substrates that may be used are described in U.S. Pat. No. 6,833,717, which issued Dec. 21, 2004, entitled “Electron Beam Test System with Integrated Substrate Transfer Module,” which is incorporated herein by reference to the extent it is not inconsistent with this disclosure. Many of the components may similarly be used for a writing system.

The substrate 705 may be in continuous motion during writing or test, or the substrate may be moved incrementally during the writing or test sequence. In this manner, the entire substrate 705 may be patterned or tested in one travel path in the chamber 710. Once the sequence is complete, the chamber 710 may be vented, and the substrate 705 may be transferred to the load lock chamber 725 for subsequent return to ambient environment.

While the foregoing is directed to embodiments of the invention, other and further embodiments of the invention may be devised without departing from the basic scope thereof, and the scope thereof is determined by the claims that follow.

We claim:

1. A method of patterning a layer on a large area substrate, comprising:
providing a large area substrate having a photoresist layer;
emitting a plurality of charged particle beams from charged particle emitters, wherein each charged particle beam has an emitted beam current of at least I₀; and
guiding each charged particle beam onto the photoresist layer on the large area substrate, wherein the beam current on the photoresist layer is at least 0.4 I₀, and each charged particle beam is deflected and switched on and off to generate an exposed pattern on the photoresist layer.

2. The method according to claim 1, wherein a different one of the charged particle beams is guided and deflected to scan each one of multiple regions of the photoresist layer.

3. The method according to claim 1, wherein the exposed pattern on the photoresist layer on the large area substrate corresponds to a pattern for a black matrix layer for a color filter.

4. The method according to claim 1, wherein the area of the large area substrate is at least 40000 cm².

5. The method according to claim 4, wherein all areas to be patterned on the large area substrate are exposed within 7 minutes or less;

6. The method according to claim 4, wherein the shape of the large area substrate is rectangular.

7. The method according to claim 2, wherein each of the multiple regions is at least 280 mm by 280 mm.

8. The method according to claim 7, wherein the multiple regions are scanned by deflecting the charged particle beams in two dimensions.

9. The method according to claim 7, wherein the multiple regions are scanned by deflecting the charged particle beams in one dimension and moving the large area substrate in a second dimension.

10. The method according to claim 1, wherein the beam current of each charged particle beam on the photoresist layer is at least 0.8 I₀;

11. A method of manufacturing a color filter, comprising:
providing a large area substrate with a black matrix layer for a color filter in a chamber of a charged particle beam system;
exposing a photoresist on the black matrix layer with the charged particle beam system, wherein less than 14 charged particle beams are scanned over the photoresist;
etching recesses in the black matrix layer; and
filling color filter materials corresponding to colors of the color filter in the recesses etched in the black matrix layer.

12. The method according to claim 11, further comprising:
deflecting the charged particle over the color filter;
detecting the backscattered or secondary charged particles with a plurality of detectors, wherein each detector corresponds to one charged particle beam and generates a detector signal; and
evaluating the detector signal of each detector to inspect the color filter.

13. The method according to claim 11, further comprising:
emitting the charged particle beams from a plurality of charged particle emitters, wherein each charged particle beam has an emitted beam current of at least I₀; and
guiding each charged particle beam onto the photoresist layer, a beam current on the photoresist layer is at least 0.4 I₀.

14. The method according to claim 13, wherein the number of charged particle beams is 8 or 10.

15. The method according to claim 14, wherein the area of the large area substrate is at least 40000 cm².

16. A charged particle beam system for manufacturing devices on a large area substrate, comprising:
a chamber for receiving the large area substrate;
a plurality of charged particle beam generators arranged in an array and fixed to the chamber; and
a plurality of charged particle beam guiding optics, each corresponding to one of the charged particle beam generators and each having an emitter, a grid, an anode with an aperture and at least two lenses, wherein each of the charged particle beam optics is configured to deliver at least 40% of the charged particles emitted from the emitter to the large area substrate.

17. The charged particle beam system according to claim 16, wherein the number of charged particle beam generators is 8 or 10.

18. The charged particle beam system according to claim 17, wherein the area of the large area substrate is at least 40000 cm².

19. The charged particle beam system according to claim 18, wherein each charged particle beam generator has a footprint area of at least 280 mm by at least 280 mm

20. The charged particle beam system according to claim 16, wherein each of the charged particle beam optics is configured to deliver at least 80% of the charged particles emitted from the emitter to the large area substrate.

21. The charged particle beam system according to claim 16, wherein each charged particle beam generator comprises a detector for detecting a signal for inspecting the large area substrate.
